(19) United States
(12) Patent Application Publication
(10) Pub. No.: US 2002/0072706 A1
(43) Pub. Date: Jun. 13, 2002

(54) Transluminal drug delivery catheter

(76) Inventors: Thomas Hiblar, Everett, WA (US); Lucas S. Gordon, Sammamish, WA (US)

Correspondence Address: Ronald M. Anderson, LAW OFFICES OF RONALD M. ANDERSON, Suite 507, 600 - 108th Avenue N.E., Bellevue, WA, 98004, US

(21) Appl. No.: 09/989,030

(22) Filed: Nov. 21, 2001

Related U.S. Application Data
(60) Provisional application No. 60/254,938, filed on Dec. 11, 2000.

(51) Int. Cl. A61M 29/00

(52) U.S. Cl. 604/101.01; 604/101.03

(57) ABSTRACT

A catheter for delivering a medical agent to tissue external to a body passage through which the catheter is inserted. The catheter includes a tissue piercing member, e.g., a needle having a rectangular cross-section that is asymmetrically flexible, such that the member is substantially more flexible about a first cross-sectional axis than about a second cross-sectional axis. The tissue piercing member is thus readily deflected about one axis, away from the catheter and into the tissue, while minimizing buckling of the tissue piercing member about the other axis. The catheter can include a balloon located proximal to and on the same side as the piercing member, and a second balloon located distal to and on the opposite side as the piercing member. When inflated, the balloons cause the portion of the catheter between the balloons to flex and bend, facilitating penetration of the tissue by the tissue piercing member.

### RELATED APPLICATIONS

This application is based on prior copending provisional application Serial No. 60/254,938, filed on Dec. 11, 2000, the benefit of the filing date of which is hereby claimed under 35 U.S.C. § 119(e).

### FIELD OF THE INVENTION

The present invention is generally directed to the delivery of medical agents through a body passage, and more specifically, to the delivery of therapeutic and diagnostic agents through the wall of a vascular conduit where the agents are too large to diffuse through the walls of adjacent capillary blood vessels into the surrounding interstitial tissue.

### BACKGROUND OF THE INVENTION

It is often necessary to deliver therapeutic and diagnostic agents through the wall of a vascular vessel and into the interstitial tissue of a patient. Such medical agents include antibiotics, chemotherapy agents, proteins, DNA, stem cells, and even conjugated particles or drug delivery vessels such as liposomes. Delivery of low molecular weight medical agents into interstitial tissue is easily accomplished due to the permeability of the capillary and venule blood vessels to small molecules. Typically, such drugs are administered by intravenous injection. The injected drug is carried throughout the body with the patient's blood and passes into all tissues having sufficiently permeable capillary blood vessels. Unfortunately, drugs delivered in this manner may reach many locations in a patient's body where the drug is not desired. Accordingly, it would be desirable to provide a drug delivery system that more specifically targets the portions of the body that should receive the drug being administered.

The endothelial cells that line the inner walls of most capillary vessels have a spacing of approximately 8 nanometers, allowing molecules smaller than 8 nanometers to pass into the interstitial tissue beyond the wall. This permeability effectively limits drug delivery across the capillary endothelium to smaller molecules having a mass of less than about 10,000 Daltons. Several technologies have been developed to increase the permeability of capillaries, including vasoactive drugs that increase endothelial cell spacing and electroporation, to effectively increase the rate of drug delivery across the endothelium. However, it is still very difficult to deliver large molecules and cellular agents into the vessel wall or into the interstitium beyond, without some type of physical puncturing device.

Furthermore, in some cases it is desirable to deliver a medical agent into the interstitial space surrounding a vessel that is impermeable to a molecule of any weight. Such vessels include arteries and veins, neither of which allow transfer of an agent into the interstitial tissue surrounding them. Local intramural delivery devices address this problem by contact or penetration of the vessel wall and drug delivery to that site.

While several catheters have been designed for delivering agents to a vessel, none of them have successfully incorporated all of the following desirable features: simplicity of use, minimal systemic drug delivery, and a simple, reliable mechanical design. For example, one catheter is disclosed by Helzel in U.S. Pat. No. 4,861,336 as a puncture catheter for piercing the vena cava to gain access to the portal vein. This fluid communicating path is then stabilized with a porto-caval shunt. While the catheter described is useful for making this connection, it is neither sufficiently small nor flexible enough to be readily inserted and used in small, and often torturous, arteries and veins.

U.S. Pat. No. 5,354,279 (Hofling) discloses a catheter for injection of a fluid into body cavities or hollow organs, which includes a plurality of hollow, round needles within the catheter shaft. Retraction of a catheter head causes the needles to project from the catheter head through openings proximate the distal end of the catheter assembly. In one embodiment, a balloon is disposed on the catheter in order to dilate any plaque that may be present within the vessel. Once the needles penetrate into the wall of the cavity, the fluid is injected into the wall. Movement of a plurality of needles through a relatively long intravascular catheter typically would be subject to significant drag between the needles and the catheter shaft. The Hofling design provides a catheter that has low retraction friction forces, since the needles do not slide relative to the catheter shaft. Unfortunately, the Hofling catheter includes many elements, resulting in a catheter that is too stiff and bulky to navigate a torturous path through blood vessels. Additionally, the vessel wall piercing force is generated only by the stiffness of the pre-curved needles, limiting the piercing force that can be applied by the small needles. This problem is significant if the catheter is placed within a vessel that is significantly larger than the catheter diameter, because as the thin needles extend beyond the stabilizing catheter shaft, they experience buckling forces as they attempt to penetrate the vessel wall. This problem is especially pronounced if the vessel is thick and muscular, as is often the case in large arteries. Even if the needle successfully pierces such a vessel wall, it is likely to curve in an unpredictable and undesirable manner, due to the buckling instability of a round needle under the applied longitudinal force.

This buckling instability is partially addressed by Faxon et al., in U.S. Pat. No. 5,464,395. The Faxon device is a catheter incorporating a lumen passage with a round needle cannula slidably disposed within it. It also includes a lumen for advancing the catheter over a pre-positioned guide wire, as is known in the art. The catheter is advanced over the guide wire within an artery while the needle is in a retracted position within a lumen of the catheter. The tissue piercing tip of the needle is normally curved so that it will exit the distal opening of the catheter lumen at an angle between 30° and 90°. Advancing the needle pushes the curved and sharpened tip outward from the catheter and into the artery wall. However, when in the retracted position, the curve of the needle has a tendency to flex the catheter into that same curve, unless the catheter shaft is stiffer than the needle. Thus, the catheter either is caused by the needle to have a curve at the distal end, making navigation and guide wire tracking more difficult, or the catheter shaft itself is sufficiently stiff to resist the curving force from the needle cannula, which again causes difficulty in navigation and advancement of the catheter along a tortuous path over the guide wire.

The Faxon catheter includes an asymmetrically disposed balloon that is fixedly and sealably secured to the distal portion of the shaft on the opposite side as the exit orifice provided for the tip of the needle cannula. Inflation of the balloon brings the distal portion of the catheter into direct contact with the artery wall. This configuration reduces the buckling tendency of the needle as it pierces the wall by shortening the distance between the exit orifice in the catheter and the vessel wall. However, because the needle is round, it may still curve in any direction as it extends further from the catheter exit orifice. This problem limits the user's ability to accurately target the needle tip in interstitial tissue at a distance from the vessel puncture location. Additionally, because the balloon does not expand against the entire inner circumference of the vessel, it provides limited stability during needle advancement through the vessel wall.

The balloon described by Faxon is attached by adhesive or heat bonding, making it very difficult to attach a balloon so as to provide complete, or nearly complete (360°) contact with the vessel wall, if the balloon is proximate to the needle exit orifice. This difficulty is due to the position of the needle exit orifice being located at the balloon to shaft seal. Faxon discloses a configuration wherein a balloon providing full wall contact is positioned distal to the needle cannula. While providing excellent sealing of the balloon to the catheter shaft, this configuration lacks adequate direct support for the needle during its extension into the vessel wall.

Neither Faxon, nor any of the previously described catheters, address the need to provide torque control or visual guidance of the catheter shaft for precise rotational positioning of the catheter tip. Thus, the methods and devices used in the prior art to deliver medical agents into or beyond the wall of a vessel passage using a catheter advanced needle have several deficiencies relating to flexibility, trackability, torqueability, and the precise targeting of the agent administered to the vessel wall or interstitial tissue.

### SUMMARY OF THE INVENTION

The present invention is directed to a method and apparatus for delivering a medical agent from within a body passage, such as a blood vessel. In accord with the present invention, a medical agent delivery device is defined that can readily be advanced through small and tortuous passages and precisely positioned to deliver a medical agent to a desired location. In general, the delivery device is configured as a catheter that includes an elongated shaft having at least one lumen through which an elongated tissue piercing member is advanced. The tissue piercing member is sufficiently flexible that the catheter is readily advanced through small, tortuous vessels, yet is also sufficiently rigid as to enable penetration into tissue around a vessel.

Preferably, the tissue piercing member is asymmetrically flexible, such that the member is substantially more flexible about a first cross-sectional axis than it is about a second cross-sectional axis that is substantially different than the first cross-sectional axis. Such a characteristic enables the piercing member to be readily deflected away from a longitudinal axis of the catheter and into tissue outside the vessel, while minimizing any buckling that would tend to inhibit the piercing member from piercing the wall of the vessel or adversely affecting the aim of the piercing member. A piercing member having a rectangular cross-sectional shape can be beneficially employed, as well as other shapes that are not symmetric in all directions. Preferred cross-sectional shapes that provide such flexibility include those having a width along a first orthogonal axis that is substantially greater than a width along a second orthogonal axis.

An asymmetrically flexible tissue piercing member can also be provided that has a cross-sectional shape that is symmetric relative to two orthogonal cross-sectional axes. For example, a square shaped tissue piercing member can be asymmetrically flexible if adjacent sides are fabricated to have different flexibility. Use of such a tissue piercing member in a catheter provides a device having sufficient flexibility such that it is readily advanced through small vessels, yet which is sufficiently rigid as to enable penetration into tissue adjacent to the vessel.

Another aspect of the present invention ensures that the distal end of the catheter can be accurately positioned within a body passage. Specifically, the shape of the tissue piercing member relative to a lumen through which the tissue piercing member moves within the catheter ensures that the tissue piercing member cannot freely rotate within the lumen, thereby enhancing a level of control a user has in determining a location at which the tissue piercing member will penetrate a wall of the body passage.

Still another aspect of the present invention is realized by ensuring that the cross-sectional size and shape of the tissue piercing member and the lumen through which the tissue piercing member moves are selected so that friction between the tissue piercing member and the lumen is reduced and by minimizing the points of contact between the tissue piercing member and the walls of the lumen.

To ensure that the tissue piercing member is properly positioned for good penetration into the wall of the body passage, an inflatable balloon can be disposed on the catheter opposite an exit orifice in the lumen through which the tissue piercing member passes to reach the wall of the body passage. When inflated, the balloon both stabilizes the catheter within the body passage, and firmly biases the exit orifice of the lumen against the wall of the body passage, ensuring that the tissue piercing member is properly disposed to readily pierce the body passage wall. Another element enhancing penetration of the body passage wall by the tissue piercing member is a ramp that is formed in the lumen of the catheter and disposed adjacent the exit orifice. The ramp deflects the tissue piercing member toward the body passage wall, at an angle closer to perpendicular to the longitudinal axis of the catheter than would be possible without the ramp. The use of such a ramp enables good penetration of the body passage wall to be achieved without requiring that the tissue piercing member be pre-curved, since use of a pre-curved tissue piercing member can make the catheter difficult to advance through tortuous vessels.

Alternatively, two balloons, respectively disposed distally and proximally to the exit orifice in the lumen, can be employed. When inflated, these balloons isolate a portion of the body passage, so that no medical agent can migrate either proximally or distally, relative to the exit orifice in the lumen through which the tissue piercing member passes. When the tissue piercing member is a solid wire, rather than a needle that includes its own lumen, the wire is advanced through the exit to create a channel that passes through the body passage wall into tissue beyond. When the wire is removed, a medical agent is introduced via the lumen in the catheter, filling the volume in the body passage isolated between the two balloons and flowing through the channel into tissue beyond the body passage. Also, the two balloons can instead be disposed on opposing sides of the catheter, forcing the catheter to bend, thereby ensuring that the tissue piercing member enters the wall of the body passage at an angle more nearly perpendicular to a longitudinal axis of the body passage than would otherwise be possible. In another embodiment of the present invention, one of the balloons is formed integral to a shaft of the catheter, to minimize the number of joints that could leak.

A still further aspect of the present invention relates to a method for delivering a medical agent to a desired location within a patient's body, the steps of which are generally consistent with the functions provided by the elements of the apparatus described above.

### BRIEF DESCRIPTION OF THE DRAWING FIGURES

The foregoing aspects and many of the attendant advantages of this invention will become more readily appreciated as the same becomes better understood by reference to the following detailed description, when taken in conjunction with the accompanying drawings, wherein:

FIG. 1 is a schematic side view of an embodiment of a catheter system in accord with the present invention, showing a piercing needle in a distally extended position, and a fully inflated distal eccentric balloon;

FIG. 2 is an enlarged, cross-sectional view of the distal end of the catheter of FIG. 1;

FIG. 3 is a cross-sectional view of the distal end of the catheter, taken along section line 3-3 in FIG. 2;

FIG. 4 is a schematic side view of an embodiment of a catheter system in accord with the present invention that includes two distal eccentric balloons, disposed within a vein;

FIG. 5 is an enlarged side view of the distal end of the catheter of FIG. 4, showing the balloons inflated and a piercing member in a retracted position;

FIG. 6 is an enlarged side view of the distal end of the catheter of FIG. 5, showing the balloons inflated and the piercing member in a distally extended position, during drug delivery into tissue surrounding the vein;

FIG. 7 is an enlarged, cross-sectional view of the distal end of yet another embodiment of a catheter in accord with the present invention, showing an opposed pair of inflated balloons, and a piercing member in a distally extended position, during drug delivery into tissue surrounding the vein;

FIG. 8 is a side view illustration of a distal portion of yet another embodiment of a catheter in accord with the present invention, showing an inflated balloon, a needle in a distally extended position, and an extended ramp that deflects the needle in a desired direction;

FIG. 9 is a partial cutaway cross-sectional view of a needle disposed within a lumen of a catheter shaft, the lumen having a shape designed to minimize frictional forces between the needle and the lumen;

FIG. 10, is a similar view of a different embodiment than that shown in FIG. 9, wherein a differently shaped lumen and needle that similarly reduce frictional forces between the needle and the lumen are employed;

FIG. 11A (Prior Art) is a cross-sectional view of a piercing member that is symmetrically flexible about any cross-sectional axis; and

FIGS. 11B-11D are cross-sectional views of different embodiments of piercing members that exhibit asymmetrical flexibility about different cross-sectional axes, in accord with the present invention.

### DESCRIPTION OF THE PREFERRED EMBODIMENT

Overview

The present invention is used for delivering a medical agent to tissue external to a body passage, such as a blood vessel, by advancing a catheter to a desired position within the body passage. The catheter includes an elongated tissue piercing member, whose construction or cross-sectional shape provides sufficient flexibility such that it doesn't restrict the catheter from being readily advanced through small, tortuous vessels, yet is sufficiently rigid to enable penetration into tissue outside the body passage. In one embodiment, the shape of the tissue piercing member relative to a lumen through which the tissue piercing member moves within the catheter ensures that the tissue piercing member cannot freely rotate within the lumen, thereby enhancing a level of control that a user has in determining a location where the tissue piercing member will penetrate a wall of the vessel. Further embodiments, as described in detail below, include one or more balloons disposed on the catheter to enhance the ability of the tissue piercing member to penetrate tissue, a lumen having a cross-sectional size and shape to reduce friction with the tissue piercing member, and a ramp provided in the catheter to direct the tissue piercing member outwardly into the wall of the vessel.

Definition of Terms

Before explaining how these features are implemented, it will be helpful to define several terms. Note that the definitions provided below are applicable both to this disclosure and to the claims that follow.

A “catheter” refers to a tubular, flexible instrument that is inserted into a patient's body for withdrawing or introducing fluids, or performing diagnostic or therapeutic procedures, and which is usable within a duct, a blood vessel, a hollow organ, or a body cavity—all of which are encompassed within the term “body passage” as used herein and in the claims.

A “desired tissue location” refers to a site in a patient's body where a medical agent is to be locally delivered. The desired tissue location may be tissue comprising the inner, middle, or outer wall of a body passage, or tissue external to the wall of the body passage. Once the general desired tissue location has been selected, knowledge of the vascular, lymph, or bile system at that location will permit the user to select an appropriate access site and a catheter of the proper dimension that can be advanced through a vein, artery, bile duct, lymph duct, or other anatomical body passage in proximity to the desired tissue location.

The term “medical agent” encompasses therapeutic agents, diagnostic agents, imaging agents, cellular agents, and other agents that are of medicinal value. A medical agent may also comprise conjugated agents, incorporating functional components including carriers (such as microparticles) or encapsulating agents (such as liposomes), therapeutic agents (such as drugs or prodrugs), imaging agents (such as radioactive components), and targeting agents (such as antibodies). Without implying any limitation of the present invention, exemplary examples of carriers and encapsulants include polymer solids, polymer gels, niosomes, and microscopic bubbles.

The term “therapeutic agent” refers to any drug, chemical or other material that might be infused to an internal treatment site within a patient's body, or used in the treatment of a disease or disorder. Examples, without limitation, include gene therapy agents, antibiotics, chemotherapy agents, anti-neoplastics, hormones, antivirals, radiation (via radiation sources such as cobalt, radium, radioactive sodium iodide, etc.), anticoagulants, enzymes, hepatoprotectants, vasodilators, prodrugs, and the like. A therapeutic agent may also be combined with another liquid such as physiologic saline or the like, and may be administered using the apparatus and methods described herein in accord with the present invention.

The term “diagnostic agent” refers to any chemical or other material that is used to detect or to determine the nature of a disease or disorder. Exemplary examples of diagnostic agents include, without implying any limitation of the present invention, dyes that react with metabolic products of a particular disease, and radioactive materials that bind to and thereby indicate the presence of disease-causing entities within a patient's body.

The term “imaging agent” refers to any material comprising an agent that is employed with various types of body scanners to more readily distinguish a specific tissue from surrounding tissues. Examples, without limitation, include radiopaque contrast agents imaged by X-ray systems; ferromagnetic or superparamagnetic metal particles imaged by magnetic resonance; and gas bubbles, low density spheres, and hollow spheres imaged by ultrasound.

The term “cellular agent” refers to a biological cellular structure, either living or non-living. Examples of cellular agents include viruses, bacteria, autologous, homologous, or interspecies cells such as stem cells, immune cells such as T cells, natural killer cells, B cells, monocytes, lymphokine-activated killer cells, tumor-infiltrating lymphocytes, lymph node lymphocytes, endothelial cells, hepatocytes, antigen-presenting cells, islet cells, and the like.

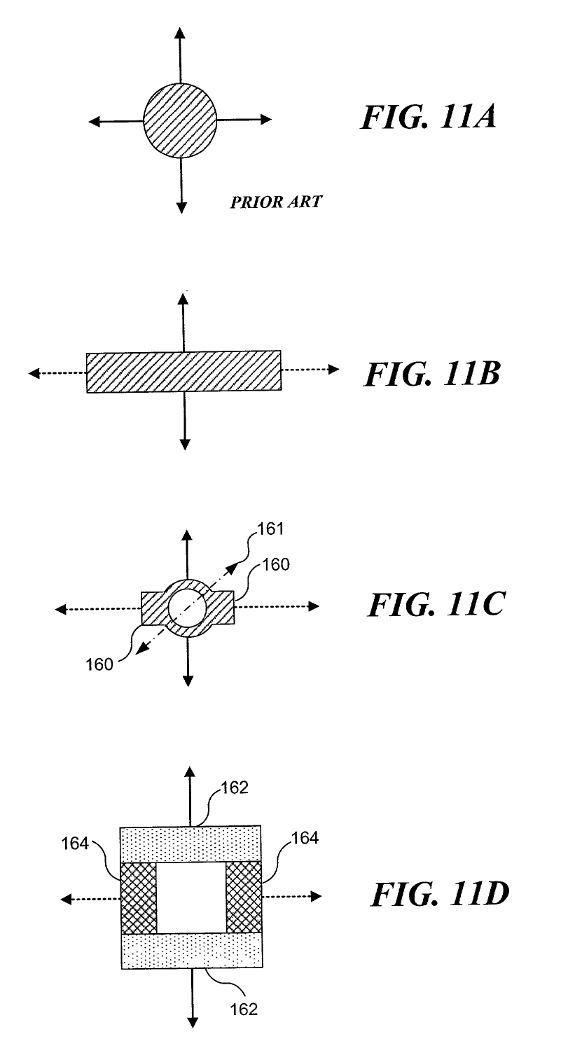

The term “asymmetrical flexibility” indicates an element that has different levels of flexibility about different cross-sectional axes. FIG. 11A (described in detail below) illustrates an element exhibiting symmetrical flexibility, while FIGS. 11B-11D (also described in detail below) illustrate examples of elements exhibiting asymmetrical flexibility.

The term “axially symmetrical” indicates an element that is symmetrical about any cross sectional axis, whereas the term “axially asymmetrical” indicates an element that is symmetrical about less than all cross sectional axes. Both a square and a circle are axially symmetrical, thus both FIGS. 11A and 11D (described in detail below) illustrate shapes exhibiting axial symmetry. FIGS. 11B and 11C (also described in detail below) illustrate examples of shapes exhibiting axial asymmetry.

The term “about” means that the characteristic modified by such term may vary by zero to 20 percent from the nominal value for that characteristic, and still be within the scope of this invention, unless expressly stated to the contrary.

Catheter Design Considerations

A catheter designed for delivery of a medical agent into the wall of a body passage or beyond into a desired tissue location, must meet certain requirements. These requirements include the ability to traverse and navigate through small and torturous body passages or vessels, often at a substantial distance from an initial insertion point into the body. A catheter preferably has sufficient flexibility to follow or track over a thin guide wire that is inserted a patient's body and advanced to a desired location and then used to define the path and form a guiding rail for advancing the catheter to the desired location. Having been advanced to the desired location, the catheter must be manipulated to orient the drug delivery mechanism disposed at the distal end, into the proper orientation relative to the body passage into which the catheter has been inserted. To enable accurate control of the distal end of the catheter, the catheter must be able to accurately transmit torque from the proximal end to the distal end, with repeatability and precision, and with a minimal amount of windup or twisting, as the catheter shaft is rotated. If the catheter shaft experiences windup, it will tend to suddenly release that twist in a rapid, uncontrolled manner (referred to by clinicians as whipping), making it almost impossible to control the final rotational orientation of the distal end of the catheter. These requirements dictate that a suitable catheter possess both longitudinal flexibility and rotational stiffness. If a the drug delivery mechanism includes a needle (or wire) that is used to penetrate the vessel wall, it is also desirable to maintain the correct orientation and stability of the needle as is extends beyond the end of the catheter. If the needle is too stiff, it will adversely affect the tracking of the catheter tip over the guide wire, and if too flexible, it will tend to buckle, so that the wall of the body vessel is not penetrated or so that the aim is deflected to an undesired location.

Preferred embodiments of the current invention meet all of these requirements by employing several novel elements, including a needle having an asymmetrical flexibility relative to its cross-section, and various means to position the needle tip securely against the vessel wall during penetration. In at least one embodiment, the asymmetrical flexibility is provided by a piercing element whose cross sectional shape is axially asymmetrical, while in at least one additional embodiment, the asymmetrical flexibility is provided by a piercing element whose cross sectional shape is axially symmetrical. The present invention also enables the user to maintain visibility of both the longitudinal and rotational position of the distal end of the catheter and the needle during use, with a minimum number of additional parts, by combining functions of many of the components of the catheter. Thus, a mechanically robust and simple catheter is achieved. Embodiments of the present invention eliminate joints and bonds found in prior art catheters, to achieve a more robust structure than is typical in prior art catheters used to administer a medical agent to a desired site.

Exemplary Preferred Embodiments

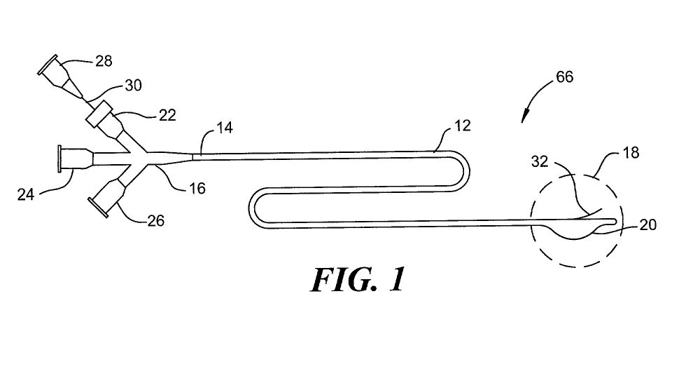

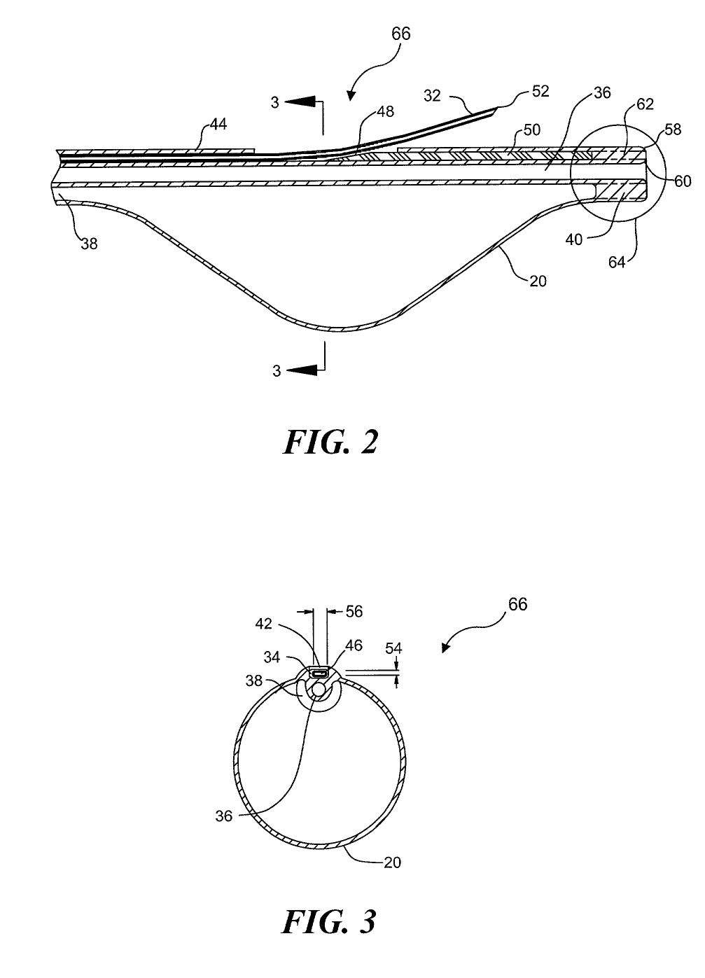

Referring to FIGS. 1-3, an exemplary catheter assembly 66 includes an elongated shaft 12, having a proximal end 14 attached to a fitting 16, and a distal end 18 that includes an eccentrically disposed inflatable balloon 20. Fitting 16 has a piercing needle port 22, a guide wire port 24, and a balloon inflation luer port 26. An elongated piercing needle 30 enters catheter 66 through piercing needle port 22, a distal end 32 of piercing needle 30 exits obliquely from distal end 18 of the catheter, at a location opposite inflatable balloon 20. A drug infusion luer port 28 is attached at the proximal end of piercing needle 30, and is in fluid communication with a lumen 46 (FIG. 3) that runs through the length of piercing needle 30.

A syringe (not shown) containing a medical agent (such as a therapeutic agent) to be injected into a patient's tissue may be attached to drug infusion luer port 28. Catheter 66 is of a length sufficient to be introduced into a suitable vessel, such as a femoral vein, and to extend to a desired vein, such as a coronary vein, in proximity to a desired tissue location within a patient. Preferably, catheter 66 is constructed of suitable materials and is of a cross-sectional size that will provide adequate flexibility to negotiate a path through coronary veins that may be curving and torturous.

Catheter shaft 12 includes a piercing needle lumen 34, a guide wire lumen 36, and a balloon inflation lumen 38, each extending from catheter shaft proximal end 14 to catheter shaft distal end 18. Guide wire lumen 36 is preferably circular to accommodate a round guide wire (also not shown), while piercing needle lumen 34 is rectangular to accommodate a generally rectangular piercing needle 30. Piercing needle lumen 34 intersects an orifice 42 in a wall 44 of catheter shaft distal end 18. Orifice 42 is sized to allow piercing needle distal end 32 to exit obliquely from lumen 34 and is located opposite from balloon 20. Piercing needle distal end 32 has an angled, sharp tip 52, to engage and pierce the vessel wall (not shown in these figures) and to facilitate penetration through that vessel wall.

Balloon inflation lumen 38 is preferably crescent shaped, thereby enabling balloon 20 to be formed integrally with catheter shaft 12. Balloon catheters typically are fabricated by adhesively or thermally bonding the ends of a separate balloon onto a catheter shaft. If a separate, eccentric balloon were used in this embodiment of the present invention, it would similarly require an additional bond between the balloon and the catheter shaft around orifice 42. Maintaining such a bond, without leakage, while inflating the balloon, is very difficult. By forming eccentric balloon 20 integrally with shaft 12, multiple bonds between the balloon and the catheter shaft are eliminated, along with the possibility of leakage from such bonds. Because balloon 20 is integrally formed with catheter shaft 12, a material must be selected that the requirements for both elements. Suitable materials include polyurethane, cross-linked polyethylene, polyamide, or preferably a polyether block amide such as PEBAX®.

Piercing needle 30 is slidably disposed in piercing needle lumen 34, and its distal end 32 is outwardly deflected by an inclined slope 48 on a proximal end of ramp pin 50, as piercing needle 30 is advanced distally to exit obliquely through orifice 42. Ramp pin 50 is an elongate element having the same cross-section size and shape as piercing needle lumen 34, and is preferably secured in position within piercing needle lumen 34 by adhesive bonding, thermal bonding, or other suitable attachment methods.

It is often necessary to rotationally position the distal end of the catheter in order to advance the piercing needle into the appropriate tissue. This function requires a catheter that has rotational sensitivity and control as well as a marker (such as a fluoroscopic agent) that indicates the rotational position of the distal end, thus enabling the proper orientation of the piercing needle to be achieved. Previous drug delivery catheters have employed substantially round needles or piercing members in substantially round catheter lumens. In such a catheter, rotating the catheter generally does not rotate the round needle, and as the catheter shaft twists during rotation, alignment is not maintained between slope 48 and angled tip 52. Consequently, there is a strong possibility that jamming will occur when the angled tip is advanced against ramp pin 50. Preferably, in the present invention, neither the needle nor the catheter shaft (lumen 34, in which the needle is slidably disposed) are round, but instead, each of these elements have corresponding cross-sectional shapes that prevent a loss of alignment by ensuring that the needle or the catheter cannot rotate independently of each other.

Note that because ramp pin 50 is fabricated with a rectangular cross-section and contains slope 48, it can also serve as a rotational fluoroscopic marker if made from a radiopaque material, or plated with such a material. A preferred material for ramp pin 50 is tungsten, because it is both radiopaque and hard, providing an excellent, smooth surface against which angled piercing needle tip 52 can readily slide outwardly from the catheter shaft.

A rectangular cross-sectional shape has an added benefit for piercing needle 30, besides preventing loss of alignment. Its relatively thin vertical (as shown in FIG. 3) thickness 54 (i.e., the shorter cross-sectional dimension), enables the needle to easily flex outward when advanced distally, yet the relatively wide horizontal (as shown in FIG. 3) width 56 (i.e., the larger cross-sectional dimension) prevents twisting, buckling, and veering as the needle penetrates the vessel wall, and advances into the interstitial tissue, thus providing more accurate aiming and placement of needle tip 52 in the desired tissue location. A suitable thickness 54 for piercing needle 30 is about 0.007 inches, and a suitable width 56 is about 0.014 inches if the needle is made from a metal such as 304 stainless steel. If constructed from a more flexible material, such as a polymer, these dimensions should be increased in order to maintain the appropriate combination of stiffness and flexibility. Needle 30 is preferably made from a suitable biocompatible, radiopaque metal such as tungsten, gold alloy, platinum alloy or palladium alloy. Other suitable materials include stainless steel or nickel-titanium alloys, incorporating a gold, platinum, or similar radiopaque coating. While a rectangular cross-section is a preferred shape, it should be noted that a specific shape is not so important, so long as a shape that is selected, regular or irregular, has a cross-section such that its dimension along a first cross-sectional axis is substantially greater than along a second cross-sectional axis that is substantially different than the first cross-sectional axis. The differences in length of such cross-sectional axes ensure that the needle will exhibit different or asymmetrical flexibility about these axes. A cross-sectional shape, such as rectangle or elongated diamond shape, has such unequal length cross-sectional axes. Thus it should be understood that many different cross sectional shapes exhibiting axial asymmetry can be beneficially employed to provide a piercing element expressing such asymmetrical flexibility. It should also be noted, as is described in more detail below, relative to FIG. 11D, that cross-sectional shapes that have equal orthogonal cross-sectional axes (such as squares and circles) can also exhibit asymmetrical flexibility, if suitably constructed.

In certain applications, it may be desirable to reduce the sliding friction between piercing needle 30 and lumen 34. This reduction in sliding friction may be by coating one or both surfaces with low friction materials such as PTFE, silicone, or polyolefin or hydrogel polymer coatings. Alternatively (or in addition), as will be described in more detail below with respect to FIGS. 9 and 10, the cross-sectional shapes of the needle and lumen can be selected so as to further reduce frictional forces between those elements.

Balloon inflation lumen 38 must be closed at its distal end 40 in order to contain fluid during inflation of balloon 20. The closure of this lumen may be accomplished by thermally forming a tip 64 on distal catheter shaft 18. Such tip formation is well known in the art and may be accomplished by a variety of methods, including thermal forming using resistance heated or induction heated dies. During this tip closing operation, distal end 62 of piercing needle lumen 34 may be closed, capturing ramp pin 50, assuring that it cannot move from its desired position adjacent to orifice 42. Additionally, radiuses 58 and 60 may be formed on the distal end of the catheter shaft, making tip 64 smooth and atraumatic to passage through a vessel.

In use, catheter assembly 66 is advanced over a pre-positioned guide wire (not shown) disposed within guide wire lumen 36, until distal end 18 is disposed at the site of desired drug delivery. In order to provide a very flexible catheter, piercing needle 30 may be partially or fully withdrawn from catheter lumen 34 as the catheter is thus positioned, thereby increasing the flexibility of catheter distal end 18, providing enhanced tracking of the catheter shaft over the guide wire. Once catheter distal end 18 is properly positioned in the desired vessel location (not shown), piercing needle 30 may be advanced distally through the needle piercing lumen, until angled tip 52 is disposed in catheter distal end 18. With needle 30 now extended through the full length of lumen 34, torque applied to fitting 16 is transferred easily to catheter distal end 18, because needle 30 has a high degree of torsional stiffness, and its non-circular cross-section cannot rotate within non-circular piercing needle lumen 34. Since ramp 48 is made of radio opaque material, an operator can readily visualize the exact rotational position of distal end 18 using an appropriate imaging system.

Balloon 20 is next inflated using a pressurized fluid inflator (not shown) attached to balloon inflation port 26. As balloon 20 is inflated, it urges orifice 42 on catheter distal end 18 firmly against the vessel wall that is opposite the balloon. Piercing needle 30 may then be distally advanced through lumen 34 and up slope 48, which deflects tip 52 out through orifice 42, into the vessel wall and beyond, as desired by the operator. A syringe (not shown) containing the desired medical agent may be attached to drug infusion luer port 28. The syringe (or other fluid delivery device) administers the medical agent through piercing needle lumen 46 and into the desired tissue location. Very little medical agent is lost (i.e., not infused into the desired tissue) during injection because balloon 20 presses catheter orifice 42 firmly against the vessel wall. Balloon 20 may then be deflated and catheter 66 removed or repositioned for administration of the medical agent at a different desired location.

Although as described this embodiment of the invention includes an axially asymmetrical (e.g., having a rectangular cross-sectional shape) tissue piercing member that extends the full length of the catheter shaft, it should be appreciated that this attribute is only required for that portion of the piercing member that is disposed and advanced from within the distal portion of the catheter shaft. In some cases, it may be desirable to fabricate the tissue piercing element by partially flattening the distal end of a round or square shaft or needle to obtain the desired axial asymmetry, and the corresponding asymmetrical flexibility, in each axis.

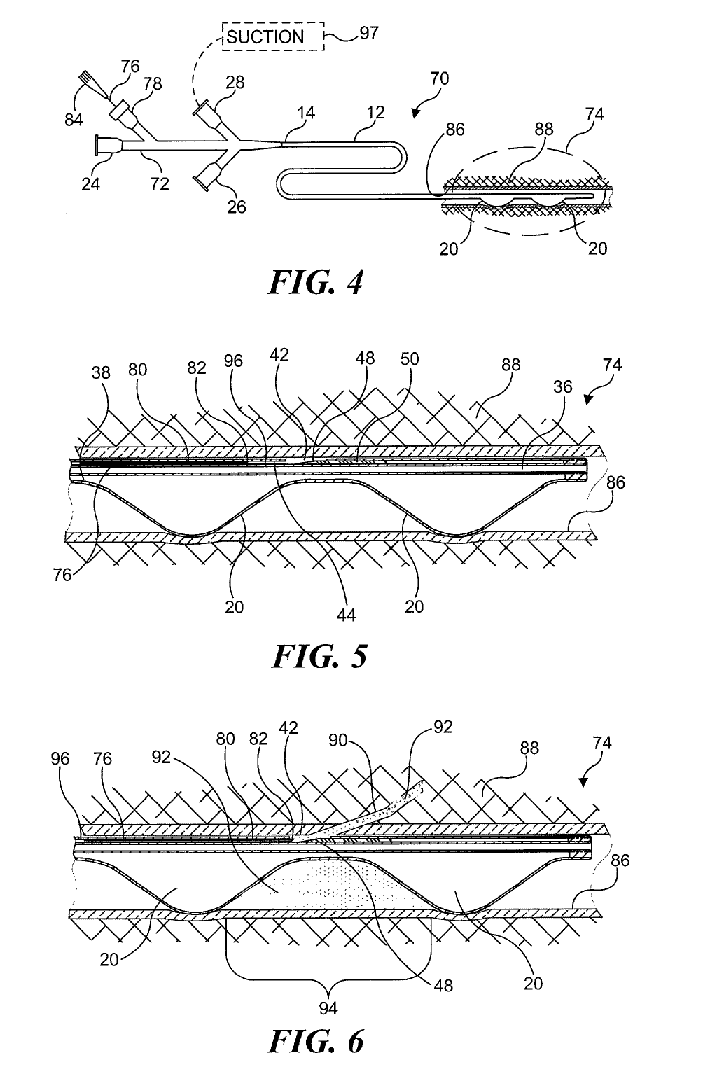

FIGS. 4-6 illustrate another embodiment of the present invention, with FIG. 4 showing the entire device, and FIGS. 5 and 6 showing only an enlarged view of the distal end of the catheter, during several steps of delivering a medical agent 92 through the wall of a vein. In these figures, those parts having the same function as in the previous embodiment are designated by the same reference numerals.

Catheter assembly 70 includes elongated shaft 12 having proximal end 14 attached to a fitting 72 and a distal end 74 having two eccentric inflatable balloons 20. Fitting 72 includes a piercing wire port 78, guide wire port 24 and balloon inflation luer port 26. An elongated piercing wire 76 enters catheter 70 through piercing wire port 78, and its distal end 80 exits obliquely from catheter distal end 74 at a location between eccentric balloons 20, and on the opposite side of the catheter shaft. Drug infusion luer port 28 is in fluid communication with a piercing wire lumen 96 of catheter shaft 12 within fitting 72. Medical agent 92, to be injected into the patient's tissue, may be administered with a syringe (not shown) when attached to drug infusion luer port 28. Note that piercing wire 76 is distinguished from piercing needle 30 in that the needle is hollow, while the wire is solid cored. Thus, the wire forms a channel when it is withdrawn, so that a medical agent can be delivered through the channel after the piercing wire is withdrawn, while the needle actually delivers the medical agent before the needle is withdrawn.

Catheter shaft 12 includes piercing wire lumen 96, guide wire lumen 36, and balloon inflation lumen 38, each extending from catheter shaft proximal end 14 to catheter shaft distal end 74. Piercing wire lumen 96 is rectangular in cross-section to accommodate the rectangular cross-sectional piercing wire 76, which has an angled and sharply pointed distal end 82. Piercing wire lumen 96 is somewhat larger than piercing wire 76 in both height and width in order to provide area for flow of the desired medical agent 92 during administration through the catheter. Piercing wire lumen 96 extends past orifice 42 in wall 44 of the catheter shaft distal end 74. Orifice 42 is sized to allow piercing wire distal end 80 to exit from piercing wire lumen 96. Piercing wire 76 is slidably disposed in piercing wire lumen 96 and its distal end 80 is bent outwardly, so that it exits from orifice 42, when as the distal end of the piercing wire is advanced distally along inclined slope 48 on the proximal end of ramp pin 50. The proximal end of piercing wire 76 is attached to a handle 84 to enable an operator to easily manipulate the piercing wire.

Catheter 70 is shown inserted percutaneously and transluminally into a patient's blood vessel 86 within a tissue 88. For example, one application of the catheter might be to deliver an angiogenic growth factor such as vascular endothelial growth factor (VEGF) into the myocardium of a patient through a blood vessel, such as the middle cardiac vein. FIG. 5 shows catheter distal end 74 positioned within vessel 86 and balloons 20 inflated, thus urging orifice 42 against the opposite wall of vessel 86.

FIG. 6 illustrates the delivery of medical agent 92 into tissue 88 after piercing wire distal end 80 has formed a channel 90 and has been withdrawn from the channel. The operator accomplishes this by advancing piercing wire pointed distal end 82 against slope 48 so that distal end 80 is directed outwardly from catheter distal end 74 through orifice 42, penetrating the wall of vessel 86 and into passing into tissue 88 beyond the vessel wall. After piercing the tissue to the desired depth, distal end 80 of piercing wire 76 is withdrawn back into piercing wire lumen 96, as shown in FIG. 6. The desired medical agent 92 is directed into drug infusion luer port 28, using a syringe (not shown) or other pressurizing means. Drug infusion luer port 28 (FIG. 4) is in fluid communication with piercing wire lumen 96, allowing medical agent 92 to flow around distal end 80 of piercing wire 76 within lumen 96 and exit through orifice 42 into the channel that the piercing wire created in the tissue. Inflated balloons 20 form a sealed segment 94 within vessel 86, thus preventing medical agent 92 from escaping from the sealed segment either proximally or distally by flowing through vessel 86. Medical agent 92 is thus forced into channel 90. After delivery of the medical agent is complete, balloons 20 are deflated and catheter 70 is removed, or moved to another location for additional drug delivery.

If it is desired to further limit the amount of medical agent 92 that enters into the patient's vascular circulation, the operator may elect to attach suction means 97 to drug infusion luer port 28, after the medical agent has been delivered into channel 90. Suitable suction means include the use of wall suction, or suction available in most hospital settings, generally provided by a centralized facility vacuum system, or individual suction pumps. Syringes or suction bulbs can also be employed. Slow deflation of balloons 20 allows blood to seep into isolated vein segment 94 as excess drug within the segment is evacuated by the suction means. Thus, balloons 20 perform three different functions, including positioning orifice 42 in a desired location within vessel 86, urging and stabilizing orifice 42 against vessel 86 during piercing wire distal end 80 penetration, and containing medical agent 92 within sealed segment 94 during delivery of the medical agent into channel 90.

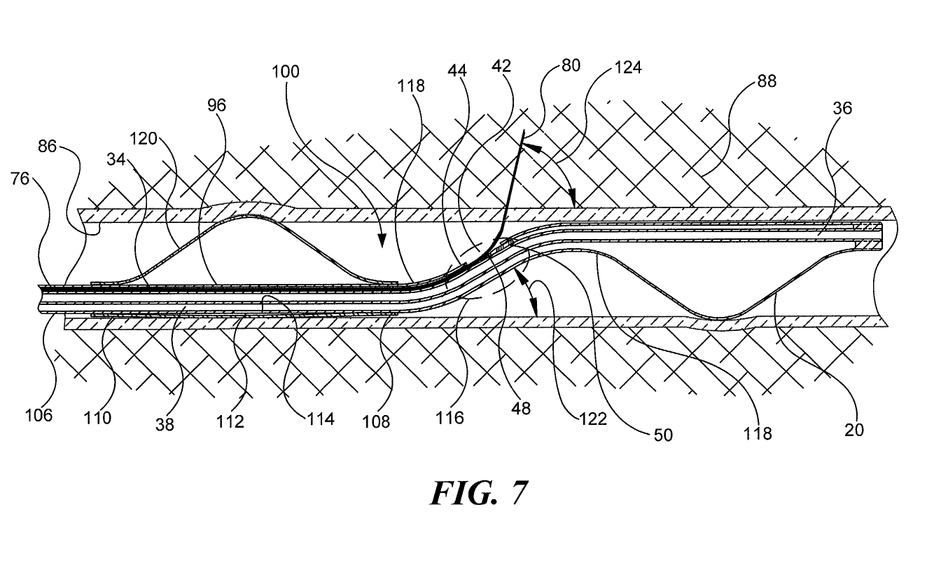

FIG. 7 illustrates yet another embodiment of the invention, showing an enlarged, cross-section of a distal end 100 of a transluminal drug delivery catheter, that is disposed within vessel 86, surrounded by interstitial tissue 88. This embodiment provides a catheter that can deliver a drug into a channel that is perpendicular or nearly perpendicular to the longitudinal axis of the catheter shaft and of the vessel, through a vessel wall, where the channel was formed by distal end 80 of piercing wire 76. As with the previous embodiment, two balloons form an isolated sealed segment during drug delivery into the formed channel. This embodiment also provides the same perpendicular (relative to the longitudinal axis of the catheter shaft and vessel) drug delivery to the tissue if a piercing needle (not shown) is used instead of a piercing wire. A catheter shaft 106 also includes piercing wire lumen 96, guide wire lumen 36, and balloon inflation lumen 38. Located on a distal end 100 of the catheter shaft are two inflatable balloons, the more distal being balloon 20, which is formed integrally with catheter shaft 106, as previously described, or formed separately and bonded to catheter shaft 106. A more proximal balloon 120 is formed separately and bonded to catheter shaft 106 by adhesive or thermal bonding, at proximal balloon end 108 and distal balloon end 110. Balloon 120 is bonded on shaft 106 in a position disposed about 180 degrees around the longitudinal axis of the catheter shaft (i.e., on the opposite side of the catheter shaft from balloon 20), thus requiring separate fabrication and attachment. Balloons 20 and 120 are inflated with a pressurized fluid delivered through balloon inflation lumen 38. Pressurization of balloon 120 from inflation lumen 38 is accomplished by an orifice 112 located in a wall 114 of catheter shaft 106. Inflation of balloons 20 and 120 result in the displacement of catheter shaft 106 against blood vessel 86, forming the catheter shaft into two reverse bends 118, and forming the portion of the catheter shaft that is thus bent at an angle 122 relative to the longitudinal axis of the straight portion of catheter shaft segment 116. Orifice 42 in wall 44 of the catheter shaft is thus positioned approximately midway between balloons 20 and 120 on catheter shaft segment 116. As in previous embodiments, extending distal end 80 of piercing wire 76 against inclined slope 48 on ramp pin 50 and through orifice 42 results in a very steep piercing angle 124 through the wall of vessel 86. This steep piercing angle is helpful in precisely delivering drugs in small or complexly disposed anatomical locations.

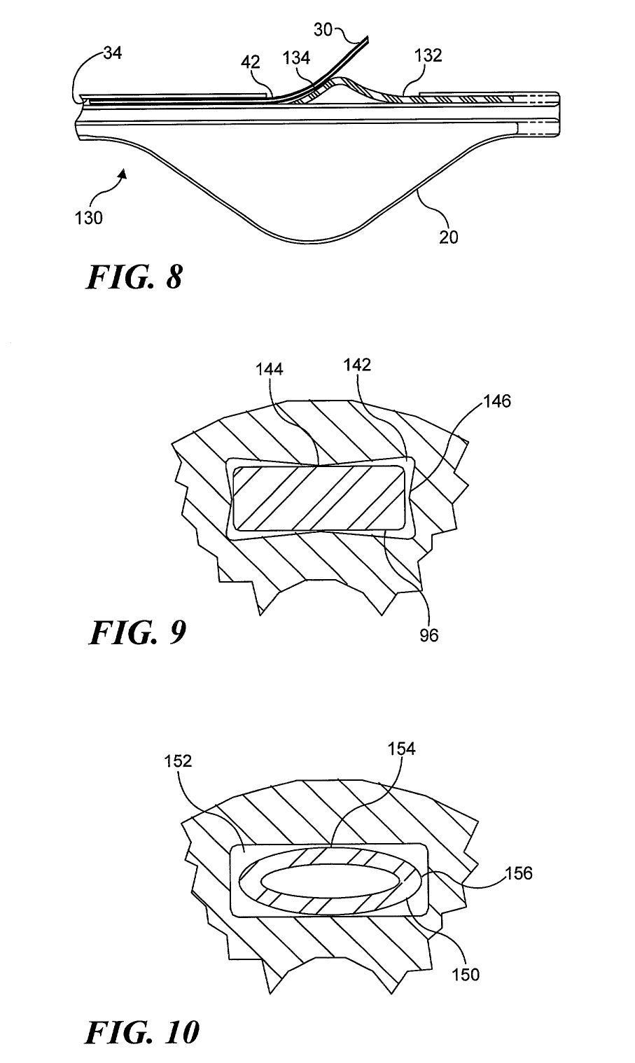

FIG. 8 illustrates yet another preferred embodiment of the invention, showing a distal portion 130 of a transluminal drug delivery catheter that includes eccentric inflatable balloon 20 and rectangular piercing needle 30 disposed within lumen 34. In this embodiment, ramp pin 132 is bonded within lumen 34, distal to orifice 42. Ramp pin 132 is formed from rectangular stainless steel wire with the substantially the same cross-sectional dimensions as lumen 34 and has a bent configuration that forms a curved, inclined slope 134, facing proximally. By curving the ramp pin in this configuration, the piercing needle may be extended from the catheter at a steeper angle than if just using the sloping end of the ramp pin to deflect the piercing needle, thereby improving the depth and accuracy of drug delivery into the interstitial tissue.

FIGS. 9 and 10 illustrate preferred embodiments designed to reduce the sliding friction between the piercing member and the catheter shaft lumen. Although both elements may optionally also be fabricated from or coated with low friction materials, it may be desirable to further reduce the friction by minimizing the surface areas between the piercing member and the lumen that are in contact with each other. FIG. 9 shows a solid rectangular piercing wire 96 within a lumen 142. All four sides of lumen 142 have a convex cross-sectional shape (i.e., are bowed inwardly) to minimize surface contact with wire 96, since the inner surface of the lumen only contacts the piercing wire at points 144 on the long sides and at points 146 on the short sides of the lumen. FIG. 10 shows an oval, hollow piercing needle 150 within a rectangular lumen 152. This combination of lumen and needle shape also effectively limits or reduces contact between the inner surfaces of the lumen and the piercing needle to points 154 on the long sides and points 156 on the short sides of the lumen.

FIGS. 11A-11D illustrate different cross-sectional shapes of piercing members, and show how flexible the piercing member is along each of two different cross-sectional axes. FIG. 11A shows a prior art piercing member that is equally flexible about two different cross-sectional axes (see arrows). FIG. 11B illustrates a piercing member having a generally rectangular cross-section, which is much more flexible about a first cross-sectional axis (solid line arrows) than it is about a second cross-sectional axis (dash line arrows). FIG. 11C shows a hollow needle having a generally circular cross-sectional lumen and with wings 160 added on opposite sides of the cross-sectional shape to stiffen the piercing member relative to bending about the axis that extends orthogonally to the axis through the wings. Note that the cross sectional shape of FIG. 11C is axially asymmetrical about an axis 161. Finally, FIG. 11D illustrates a generally square shaped cross-section of a piercing member fabricated from a first material 162 on two opposite sides that is significantly more flexible than a second material 164 from which the two other sides are fabricated. Note that the piercing member of FIG. 11D is similarly much more flexible about a first cross-sectional axis (solid line arrows) than it is about a second cross-sectional axis (dash line arrows), even though the dimensions across each of these orthogonal axes are substantially equal. Thus, different dimensions across two cross-sectional axes, while representing one way of providing the required asymmetrical flexibility, are not the only way contemplated to provide the asymmetrical flexibility.

Although the present invention has been described in connection with the preferred form of practicing it and modifications thereto, those of ordinary skill in the art will understand that many other modifications can be made to the present invention within the scope of the claims that follow. Accordingly, it is not intended that the scope of the invention in any way be limited by the above description, but instead be determined entirely by reference to the claims that follow.

The invention in which an exclusive right is claimed is defined by the following:

1. A tissue piercing catheter comprising:
(a) an elongate catheter shaft, adapted to be inserted into a body passage; and
(b) a tissue piercing member, slidably disposed within said catheter shaft, said tissue piercing member having at least a distal portion that is constructed to be asymmetrically flexible, such that said tissue piercing member is substantially more flexible about a first cross-sectional axis than it is it about a second cross-sectional axis that is substantially different than the first cross-sectional axis.

2. The catheter of claim 1, further comprising:
(a) means to extend the distal portion of said tissue piercing member that is asymmetrically flexible, from the catheter shaft and into a desired tissue location that is external to the catheter shaft, and
(b) means to deliver at least one of a therapeutic agent and a diagnostic agent to the desired tissue location.

3. The catheter of claim 1, wherein said distal portion of said tissue piercing member comprises a sharpened tip.

4. The catheter of claim 1, wherein said tissue piercing member comprises a lumen adapted to deliver at least one of a therapeutic agent and a diagnostic agent into a tissue.

5. The catheter of claim 1, wherein the distal portion of said tissue piercing member that is asymmetrically flexible comprises a composite structure, including a first material and a second material, said first material being substantially more flexible than the second material, and said first and second materials comprising different peripheral portions of said tissue piercing member.

6. The catheter of claim 1, wherein the distal portion of said tissue piercing member that is asymmetrically flexible has a cross-sectional shape such that a dimension of said cross-sectional shape along said first cross-sectional axis is substantially shorter than a dimension of said cross-sectional shape along said second cross-sectional axis.

7. The catheter of claim 1, wherein the distal portion of said tissue piercing member that is asymmetrically flexible has a cross-sectional shape that is axially asymmetrical.

8. The catheter of claim 1, wherein the distal portion of said tissue piercing member that is asymmetrically flexible has a generally rectangular cross-sectional shape.

9. The catheter of claim 1, wherein the distal portion of said tissue piercing member that is asymmetrically flexible has a generally oval cross-sectional shape.

10. The catheter of claim 1, wherein the catheter shaft comprises a lumen in which said tissue piercing member is slidably disposed, an inner surface of said lumen being coated with a material that reduces a sliding friction between the tissue piercing member and the inner surface of said lumen.

11. The catheter of claim 1, wherein the catheter shaft comprises a lumen in which said tissue piercing member is slidably disposed, said lumen having a size and a shape chosen to minimize a frictional contact with said tissue piercing member.

12. The catheter of claim 11, wherein said lumen has a generally rectangular cross-sectional shape, such that at least one side of the generally rectangular cross-sectional shape is arcuate in shape to reduce a frictional contact with said tissue piercing member.

13. The catheter of claim 1, further comprising an inflatable balloon disposed on a distal end of said catheter shaft, wherein the catheter shaft further includes a balloon lumen adapted to convey a pressurized fluid that is used to inflate said inflatable balloon.

14. The catheter of claim 13, wherein the balloon lumen has a generally crescent shaped cross-section.

15. The catheter of claim 13, wherein the inflatable balloon is formed integrally with the catheter shaft.

16. The catheter of claim 1, wherein the catheter shaft further comprises:
(a) an orifice disposed proximate a distal end of said catheter shaft;
(b) a lumen in which said tissue piercing member is slidably disposed, said lumen being in communication with said orifice; and
(c) a ramp disposed within said lumen adjacent to said orifice, said ramp deflecting said tissue piercing member outwardly from said catheter shaft as said tissue piercing member is distally advanced.

17. The catheter of claim 16, wherein said ramp extends outwardly beyond a perimeter of said catheter shaft.

18. The catheter of claim 16, wherein said ramp comprises a generally elongate member having a cross-sectional size and a shape substantially equal to a cross-sectional size and a shape of said lumen, so that a portion of said elongate member is disposed within the lumen.

19. The catheter of claim 16, wherein said ramp comprises a radiopaque material.

20. The catheter of claim 19, wherein said ramp comprises tungsten.

21. The catheter of claim 16, wherein said ramp comprises a guide channel into which said tissue piercing member is slidably directed as said tissue piercing member is distally advanced.

22. The catheter of claim 16, wherein at least a portion of said ramp is curved to form an inclined plane.

23. The catheter of claim 22, wherein said at least a portion of said ramp that is curved extends outwardly of said lumen through said orifice.

24. The catheter of claim 16, wherein said ramp is disposed flush within said lumen.

25. The catheter of claim 16, further comprising:
(a) an inflatable balloon disposed on the catheter shaft opposite said orifice, such that when the inflatable balloon is inflated, said orifice is forced by the inflatable balloon toward a wall of a body passage into which said catheter is inserted; and
(b) a balloon lumen within said catheter shaft and in fluid communication with the inflatable balloon, said balloon lumen being adapted to convey a fluid used to inflate said inflatable balloon.

26. The catheter of claim 16, further comprising a first inflatable balloon disposed adjacent to and proximal of said orifice, and a second inflatable balloon disposed adjacent to and distal of said orifice, such that when both the first balloon and the second balloon are inflated, a portion of a body passage into which said catheter is inserted, is substantially isolated.

27. The catheter of claim 25, wherein said first inflatable balloon and said second inflatable balloon are disposed opposite said orifice, such that when both the first balloon and the second balloon are inflated, said orifice is forced toward a wall of a body passage into which said catheter is inserted.

28. The catheter of claim 25, wherein said first inflatable balloon is disposed on a same side of said catheter as said orifice and said second inflatable balloon is disposed on an opposite side of said catheter from said orifice, such that when both the first balloon and the second balloon are inflated, said catheter shaft is deflected relative to a longitudinal axis of the catheter shaft, thereby enabling said tissue piercing member to more readily pierce extravascular tissue by directing said tissue piercing member at an angle more nearly perpendicular to the longitudinal axis of the catheter shaft.

29. The catheter of claim 28, wherein an extent by which the catheter is deflected about its cross-sectional axis is sufficient to enable the tissue piercing member to pierce an extravascular tissue at an angle approaching the perpendicular relative to the longitudinal axis of the catheter shaft.

30. A catheter for directly delivering a medical agent into extravascular tissue, comprising:
(a) an elongate catheter shaft, adapted to be inserted into a body passage;
(b) a tissue piercing member, slidably disposed within a lumen formed in said catheter shaft, said tissue piercing member having at least a distal portion that is asymmetrically flexible, such that said tissue piercing member is substantially more flexible about a first cross-sectional axis than it is it about a second cross-sectional axis that is substantially different than the first cross-sectional axis;
(c) means for both piercing an extravascular tissue and delivering a medical agent into an extravascular tissue;
(d) an orifice disposed in said lumen, adjacent a distal end of said catheter shaft; and
(e) a ramp disposed within said lumen such that said orifice overlaps at least a portion of said ramp, said ramp comprising a guide channel into which said tissue piercing member is slidably disposed as said tissue piercing member is advanced, thereby deflecting said tissue piercing member outwardly and away from a longitudinal axis of said catheter shaft.

31. A catheter for directly delivering a medical agent into an extravascular tissue, the catheter comprising:
(a) an elongate catheter shaft, adapted to be inserted into a body passage, said elongate catheter shaft including a lumen;
(b) a tissue piercing member, slidably disposed within the lumen in said catheter shaft, said tissue piercing member having at least a distal portion that is asymmetrically flexible, such that said tissue piercing member is substantially more flexible about a first cross-sectional axis than it is it about a substantially different second cross-sectional axis;
(c) means for both piercing extravascular tissue and delivering the medical agent into said extravascular tissue;
(d) an orifice disposed in said lumen, adjacent a distal end of said catheter shaft; and
(e) a ramp disposed within said lumen such that said orifice overlaps at least a portion of said ramp that is formed into an inclined plane, said ramp deflecting said tissue piercing member outwardly, away from a longitudinal axis of said catheter shaft as said tissue piercing member is advanced distally through the lumen, said ramp comprising a generally elongate member having a cross-sectional size and shape generally equal to a cross-sectional size and shape of said lumen.

32. A catheter for directly delivering a medical agent into an extravascular tissue, the catheter comprising:
(a) an elongate catheter shaft, adapted to be inserted into a vascular passage, said elongate catheter shaft including a lumen;
(b) a tissue piercing member, slidably disposed within the lumen of said elongate catheter shaft, said tissue piercing member having at least a distal portion that is asymmetrically flexible, such that said tissue piercing member is substantially more flexible about at least one cross-sectional axis than about other cross-sectional axes;
(c) means for both piercing extravascular tissue and delivering a medical agent into an extravascular tissue;
(d) an orifice disposed in said lumen, adjacent a distal end of said catheter shaft; and
(e) a first inflatable balloon disposed adjacent to and proximal of said orifice on a same side of said catheter as said orifice, and a second inflatable balloon disposed adjacent to and distal of said orifice on an opposite side of said catheter from said orifice, such that when both the first inflatable balloon and the second inflatable balloon are inflated, said catheter is deflected relative to a longitudinal axis of the catheter shaft, thereby enabling said tissue piercing member to more readily pierce an extravascular tissue by moving through the orifice.

33. A catheter for directly delivering a medical agent into an extravascular tissue, the catheter comprising:
(a) an elongate catheter shaft, adapted to be inserted into a vascular passage, said elongate catheter shaft having a lumen formed therein;
(b) a tissue piercing member, slidably disposed within the lumen of said catheter shaft;
(c) means for both piercing extravascular tissue and delivering a medical agent into an extravascular tissue;
(d) an orifice disposed in said lumen, adjacent to a distal end of said catheter shaft; and
(e) a first inflatable balloon disposed adjacent to and proximal of said orifice on a same side of said catheter as said orifice, and a second inflatable balloon disposed adjacent to and distal of said orifice on an opposite side of said catheter from said orifice, such that when the first inflatable balloon and the second inflatable balloon are inflated, said catheter is deflected relative to a longitudinal axis of the catheter shaft, thereby enabling said tissue piercing member to more readily pierce an extravascular tissue when distally advanced through said lumen and out through the orifice.

34. The catheter of claim 33, wherein a distal end of said tissue piercing member comprises a sharpened tip.

35. The catheter of claim 33, wherein said tissue piercing member has a lumen adapted to deliver at least one of a therapeutic agent and a diagnostic agent into an extravascular tissue.

36. The catheter of claim 33, wherein at least a distal portion of said tissue piercing member is asymmetrically flexible, such that said tissue piercing member is substantially more flexible about at least one cross-sectional axis than about other cross-sectional axes.

37. The catheter of claim 36, wherein said distal portion of said tissue piercing member that is asymmetrically flexible comprises a composite structure exhibiting different flexibility at different positions around a longitudinal axis of the composite structure.

38. The catheter of claim 36, wherein said distal portion of said tissue piercing member that is asymmetrically flexible has a cross-sectional shape such that a dimension of said cross-sectional shape along a first cross-sectional axis is substantially less than a dimension of said cross-sectional shape along a second cross-sectional axis that is orthogonal to the first cross-sectional axis.

39. The catheter of claim 36, wherein said distal portion of said tissue piercing member that is asymmetrically flexible has a cross-sectional shape that is axially asymmetrical.

40. The catheter of claim 36, wherein said distal portion of said tissue piercing member that is asymmetrically flexible has one of a generally rectangular cross-sectional shape and a generally oval cross-sectional shape.

41. The catheter of claim 33, wherein the catheter shaft comprises a lumen in which said tissue piercing member is slidably disposed, said lumen being formed so as to reduce frictional contact with said tissue piercing member by having one of a:
(a) a surface coating on an interior surface of the lumen that reduces friction; and
(b) a size and a shape that minimizes frictional contact between the interior surface of the lumen and said tissue piercing member.

42. The catheter of claim 41, wherein said lumen has a generally rectangular cross-sectional shape, and wherein at least one side of the generally rectangular cross-sectional shape is arcuate in shape, to reduce the frictional contact with said tissue piercing member.

43. The catheter of claim 33, further comprising a balloon lumen adapted to convey a pressurized fluid used for inflating said first and second balloons, said balloon lumen having a generally crescent shaped cross-sectional shape.

44. The catheter of claim 33, wherein at least one inflatable balloon is formed integrally with the catheter shaft.

45. The catheter of claim 33, wherein the catheter shaft further comprises a ramp disposed within said lumen such that said orifice overlaps at least a portion of said ramp, said ramp deflecting said tissue piercing member outwardly away from a longitudinal axis of said catheter shaft as said tissue piercing member is advanced distally through the lumen and out through the orifice.

46. The catheter of claim45, wherein said ramp extends beyond a perimeter of said catheter shaft.

47. The catheter of claim 45, wherein said ramp comprises a radiopaque material.

48. The catheter of claim 43, wherein said ramp comprises tungsten.

49. The catheter of claim 45, wherein said ramp comprises a guide channel into which said tissue piercing member is slidably disposed as said tissue piercing member is advanced.

50. The catheter of claim 45, wherein said ramp comprises a generally elongate member that is disposed at least partly within said lumen, distal to said orifice, an end of said ramp that is disposed closest to said orifice comprising an inclined plane.

51. The catheter of claim 45, wherein said ramp comprises a generally elongate member that is disposed flush within said lumen, said portion of the ramp comprising an inclined plane.

52. The catheter of claim 45, wherein said ramp comprises a curved portion that extends outwardly of said lumen through said orifice.

53. A catheter for directly delivering a medical agent into an extravascular tissue, comprising:
(a) an elongate catheter shaft, adapted to be inserted into a vascular passage, said elongate catheter shaft including a lumen; and
(b) a tissue piercing member slidably disposed within the lumen of said catheter shaft, a size and shape of both said tissue piercing member and said lumen cooperating so as to prevent said tissue piercing member from rotating within said lumen.

54. A method for accurately positioning a tissue piercing member in a desired portion of an extravascular tissue disposed proximate a body lumen, comprising the steps of:
(a) forming the tissue piercing member such that the tissue piercing member is substantially more flexible about a first cross-sectional axis, and is substantially less flexible about a second cross-sectional axis that is different than the first cross-sectional axis;
(b) including said tissue piercing member in a catheter assembly, such that said tissue piercing member is slidably disposed within said catheter assembly to be advanced through said catheter assembly;
(c) inserting said catheter assembly into the body lumen and advancing the catheter assembly through the body lumen until a distal portion of said catheter assembly is proximate the desired portion of the extravascular tissue; and
(d) advancing the tissue piercing member through the catheter assembly, until said tissue piercing member exits said distal portion of said catheter assembly proximate the desired portion of the extravascular tissue, the substantial flexibility about said first cross-sectional axis enabling said tissue piercing member to be deflected away from said catheter assembly about the first cross-sectional axis and toward said desired portion of the extravascular tissue, while the substantially lesser flexibility about said second cross-sectional axis substantially preventing buckling of said tissue piercing member, enabling the tissue piercing member to accurately penetrate the desired portion of the extravascular tissue.

55. The method of claim 54, wherein the step of advancing the catheter assembly through the body lumen comprises the steps of:
(a) sensing a position of the distal portion of said catheter assembly relative to the desired portion of the extravascular tissue; and
(b) manipulating the catheter assembly within the body passage, relative to the desired portion of the extravascular tissue, until the distal portion of said catheter assembly is disposed adjacent the desired portion of the extravascular tissue;
(c) preventing the independent rotation of the tissue piercing member within the catheter assembly; and
(d) simultaneously rotating the catheter assembly and the tissue piercing member until the tissue piercing member is disposed adjacent to the desired portion of the extravascular tissue.

56. The method of claim 54, wherein the step of advancing the tissue piercing member through the catheter assembly comprises the step of deflecting the tissue piercing member away from a longitudinal axis of said catheter assembly.

57. The method of claim 56, wherein the step of deflecting the tissue piercing member away from the longitudinal axis of said catheter assembly comprises the step of advancing the tissue piercing member along a ramp directing the tissue piercing member away from a longitudinal axis of said catheter assembly and toward the desired portion of extravascular tissue.

58. The method of claim 56, wherein the step of deflecting the tissue piercing member away from the longitudinal axis of said catheter assembly comprises the steps of:
(a) inflating a first balloon disposed adjacent to and proximal of an orifice through which the tissue piercing member extends when advanced, said first balloon being disposed on a same side of the catheter assembly as the orifice;
(b) inflating a second balloon disposed adjacent to and distal of the orifice, said second balloon being disposed on an opposite side of the catheter assembly from said orifice, the inflation of both the first and second balloons causing a portion of said catheter assembly disposed between the first and second balloons to flex and bend; and
(c) advancing the tissue piercing member along a ramp formed in the catheter assembly that directs the tissue piercing member outwardly and away from the flexed portion of said catheter assembly, through the orifice, and toward the desired portion of the extravascular tissue.

59. The method of claim 54, further comprising the step of administering a medicinal fluid into the extravascular tissue through the catheter assembly and a lumen formed in the tissue piercing member, after the tissue piercing member has pierced the extravascular tissue.

60. A method for accurately administering a medical agent to a desired portion of an extravascular tissue disposed proximate a body lumen, comprising the steps of:
(a) inserting a catheter assembly into the body lumen and advancing the catheter assembly through the body lumen, bringing a distal portion of said catheter assembly to a position that is proximate the desired portion of the extravascular tissue;
(b) inflating a first balloon disposed adjacent to and proximal of an orifice in a wall of the catheter assembly, and on a same side of the catheter assembly as the orifice;
(c) inflating a second balloon disposed adjacent to and distal of the orifice, and on an opposite side of the catheter assembly from said orifice, the inflation of both the first and the second balloons causing a portion of said catheter assembly disposed between the first and second balloons to flex and bend;
(d) advancing a tissue piercing member through a lumen in the catheter assembly until said tissue piercing member exits said distal portion of said catheter assembly through the orifice proximate the desired portion of the extravascular tissue, and pierces the desired portion of the extravascular tissue; and
(e) administering the medical agent to the extravascular tissue through the catheter assembly and through a lumen in the tissue piercing member.
